(19) United States
(12) Patent Application Publication
(10) Pub. No.: US 2008/0058426 A1
(43) Pub. Date: Mar. 6, 2008

(54) COMPOSITION AND METHOD FOR TREATING PSORIASIS

(76) Inventors: Muhammed Majeed, Piscataway, NJ (US); Subbalakshmi Prakash, Piscataway, NJ (US)

Correspondence Address: SABINSA CORPORATION, 70 ETHEL ROAD WEST, UNIT 6, PISCATAWAY, NJ, 08854, US

(21) Appl. No.: 11/616,189

(22) Filed: Dec. 26, 2006

Related U.S. Application Data
(60) Provisional application No. 60/767,572, filed on Aug. 29, 2006.

(51) Int. Cl. A61K 31/12

(52) U.S. Cl. 514/688

(57) ABSTRACT

A composition and method for treating psoriasis comprising a mixture of tetrahydrocurcumin, tetrahydrodemethoxycurcumin and tetrahydrobisdemethoxy-curcumin formulated with pharmaceutically acceptable carriers and applied to the affected skin.

This application claims the benefit of U.S. Provisional application No. 60/767,572 filed on Aug. 29, 2006, the disclosure of which is hereby incorporated by reference.

### BACKGROUND INFORMATION

Psoriasis is a common, chronic and non-infectious skin disease characterized by well defined erythematous slightly raised plaques & papules with silvery scales and typical extensor distribution.

Under normal circumstances a wound healing response leads to the generation of T lymphocytes, which further generates Tumor necrosis factor alpha (TNF-α), new blood vessels, scar tissue and epidermal proliferation.

Energy for the above processes namely ATP is generated from glycogen stores in the cells by phosphoryl kinase. In psoriatic individuals the switch-off mechanism for phosphoryl kinase is defective due to a defective Type II cAMP protein kinase linked to a defective gene. Increased phosphoryl kinase levels result in increased phosphorylation reactions, leading to the increased breakdown of glycogen stores to ATP, correlating with an increased epidermal proliferation and psoriatic activity.

Vascular Endothelial Growth Factor (VEGF) acts as a gene modifier in psoriasis and therapeutic blockade of the VEGF/VEGF receptor system is reported to represent a novel approach for the treatment of psoriasis (Young, H S et al. 2004).

Tetrahydrocurcuminoids (THC) is a colorless hydrogenated product derived from the yellow curcuminoids, the biologically active principles from the rhizomes of Curcuma longa (Turmeric), that function as efficient antioxidant compounds.

THC is a mixture of tetrahydrocurcumin, tetrahydrodemethoxycurcumin and tetrahydrobisdemethoxycurcumin.

Curcuminoids and Tetrahydrocurcuminoids (THC) are more potent antioxidants than the commonly used synthetic antioxidant, Butylated Hydroxytoluene (BHT). U.S. Pat. No. 6,653,327 describes a cross regulin composition of turmeric-derived Tetrahydrocurcuminoids for skin lightening and protection against UVB rays.

It is postulated that THC by lowering phosphoryl kinase levels in psoriatic epidermis, helps in the management of psoriasis and its associated symptoms. THC achieves this through decreasing the population of Ki-67 cells which are cells capable of dividing within the epidermis.

An important cytokine that is associated with keratinocyte proliferation in psoriasis is TNF-α. TNF-α concentrations are higher in psoriatic lesions than in unaffected skin of psoriatic patients and tend to decline with clearing of the lesions after effective therapy.

### SUMMARY OF THE INVENTION

A cream composition containing a mixture of tetrahydrocurcuminoids comprising 70-80% tetrahydrocurcumin, 15-20% tetrahydrodemethoxycurcumin, and 2.5-6.5% tetrahydrobisdemethoxycurcumin was found to be particularly effective in the treatment of psoriasis and its associated symptoms, and beneficially modified serum levels of biochemical markers (like TNF-α and VEGF) of psoriasis in psoriasis affected individuals.

### DETAILED DESCRIPTION

### Example 1

### Clinical Study Validating the Efficacy of a THC Composition in Treating Psoriasis

Study Duration: 45 days
Population: 10 psoriasis affected subjects
Intervention: THC formulated as a cream with pharmaceutically acceptable carriers applied topically three times a day for 45 days.

The primary objective in this study was to determine the efficacy and safety of the THC cream. The parameters to detect the efficacy and safety of the medication included relief from the characteristic symptoms of psoriasis like itching, erythema, scaling, infiltration, and the extent of clearance of psoriasis lesions.

Clinical improvement in the severity of psoriatic lesions as well as symptoms such as itching, erythema and scaling was observed in all subjects. Patients reported a gradual decrease in itching right from the first day of treatment.

A significant reduction in silvery scaling was observed from the 2nd week in all the subjects with visible clearance in the psoriatic lesions. A considerable reduction in erythema was found in all the subjects from week 2.

CONCLUSION: The cream formulation of THC offers relief from psoriatic lesions as evident by PASI (Psoriasis Area Severity Index) scores taken at the baseline, visit 1(3 weeks), and after 6 weeks as shown in Table 1.

TABLE 1
Comparative PASI score

| Subject No. | Baseline | Week 3 | Week 6 |
|---|---|---|---|
| 1 | 6.0 | 2.4 | 0.8* |
| 2 | 4.4 | 3.2 | 1.6* |
| 3 | 6.2 | 1.9 | 0.7* |
| 4 | 6.4 | 1.2 | 0.4* |

*Significant reduction in PASI score

TNF-α and VEGF concentrations were measured by Enzyme-Linked Immunosorbent Assay (ELISA), R and D Systems, USA. Plasma samples were analyzed for TNF-α and VEGF levels as per the protocol described by the manufacturer. The detection ranges of Human TNF-α and Human VEGF ELISA kit were 0.5 to 32 μg/ml and 31.2 to 2000 μg/ml respectively.

Significant reduction in TNF-α and VEGF concentrations was observed as shown in Table 2.

TABLE 2
TNF-α and VEGF Concentrations

| | |
|---|---|
| Plasma TNF-α | Plasma VEGF |

| | | | | |
|---|---|---|---|---|
| SUBJECT | Baseline | Final | Baseline | Final |

| | | |
|---|---|---|
| CODE | Mean (pg/ml) | Mean (pg/ml) |

| | | | | |
|---|---|---|---|---|
| 1 | 16.84 | 1.19* | 192.74 | 116.38* |
| 2 | 3.59 | 0.25* | 62.59 | 38.00* |
| 3 | 16.52 | 1.21* | 87.31 | 53.13* |
| 4 | 14.95 | 0.26* | 253.45 | 50.13* |

*Significant reduction in TNF-α and VEGF Concentration levels

The clinical outcome of this study clearly suggests the potential therapeutic use of the composition of the invention in treating psoriasis.

THC is a naturally derived composition for the treatment of psoriasis that offers comprehensive relief, and is devoid of toxic side effects unlike existing therapies.

What is claimed is:

1. A composition for the treatment of psoriasis comprising 0.01% to 5% w/w of tetrahydrocurcuminoids dispersed in a pharmaceutically acceptable carrier.

2. A composition according to claim 1 wherein the tetrahydrocurcuminoids comprise at least 95% w/w tetrahydrocurcumin.

3. A composition according to claim 1 wherein the tetrahydrocurcuminoids comprise 70-80% w/w tetrahydrocurcumin, 15-20% w/w tetrahydrodemethoxycurcumin, and 2.5-6.5% tetrahydrobisdemethoxycurcumin.

4. A method of treating psoriasis comprising applying a therapeutically effective amount of the composition described in claim 1 to the affected skin of the individual in need of treatment three times a day for a period ranging from 14 days to 90 days.

5. A method of reducing itching symptoms in psoriasis affected skin by applying to the affected area of the skin an effective amount of the composition of claim 1 three times a day for a period ranging from 1 day to 90 days.

6. A method of reducing scaling in psoriasis affected skin by applying to the affected area of the skin an effective amount of the composition of claim 1 three times a day for a period ranging from 1 day to 90 days.

7. A method of reducing serum levels of Vascular Endothelial Growth Factor (VEGF) in a psoriasis affected individual by using an effective amount of the composition of claim 1.
